(19) United States
(12) Patent Application Publication
(10) Pub. No.: US 2011/0190808 A1
(43) Pub. Date: Aug. 4, 2011

(54) Massage Assisting Tool

(76) Inventor: Conghua Li, Markham (CA)

(21) Appl. No.: 12/698,140

(22) Filed: Feb. 2, 2010

(51) Int. Cl. A61B 17/00

(52) U.S. Cl. 606/204

(57) ABSTRACT

A massage tool has a shank with a body support at one end and a massage applicator at the distal end. In use, the massage therapist applies a pressure with his/her shoulder or upper arm onto the body support while the massage applicator is contacted with the massage recipient's body and guided to perform a massage therapy. The tool is intended to reduce or prevent the stress on therapist's hands, wrists, arms, elbows and shoulders.

This application is a formal application claiming the priority of provisional U.S. patent application No. 61/148,391, filed Jan. 30, 2009, the specification of which is incorporated by reference herewith in its entirety.

### TECHNICAL FIELD

This invention relates to massage tools. More specifically, it relates to such tools that are ergonomically designed so that they can be used to massage with a reduced muscle fatigue of the massage therapist.

### DISCUSSION OF PRIOR ART

Massage services are in high demand worldwide. However, performing a massage is physically demanding both in term of physical energy as well as wear and tear of therapist's hands, wrists, arms and shoulders. Repetitive motion problems are amongst the most common.

There are many kinds of massage assisting tools. These include massage chairs, pillows, beds, mattresses, massage bath tubs, massage belts, vibrators etc.; various accessories and implements such as massage balls, massage thumps, massage rollers, etc. are also known. Examples of such products are described in the following U.S. patents and patent applications:
U.S. Pat. No. 7,431,706, issued Oct. 7, 2008 to Louis,
U.S. Pat. No. 7,252,645, issued Aug. 7, 2007 to Polins,
U.S. Pat. No. 7,179,240, issued Feb. 20, 2007 to Wu,
U.S. Pat. No. 7,141,030, issued Nov. 28, 2006 to Chen,
U.S. Pat. No. 6,267,738, issued Jul. 31, 2001 to Louis,
U.S. Pat. No. 6,241,694, issued Jun. 5, 2001 to Goulding-Thompson et al.,
U.S. Pat. No. 6,010,469, issued Jan. 4, 2000 to McAtee,
U.S. Pat. No. 5,624,385, issued Apr. 29, 1997 to Hwang,
U.S. Pat. No. 5,382,222, issued Jan. 17, 1995 to Yih-Jong,
U.S. Pat. No. 4,483,328, issued Nov. 20, 1984 to Wolocko,
U.S. Pat. No. 3,545,434, issued Dec. 8, 1970 to Woodruff,
U.S. Pat. No. 5,868,689, issued Feb. 9, 1999 to Faroky et al.,
U.S. Pat. No. 5,843,005, issued Dec. 1, 1998 to Chubinsky,
20050159689, issued Jul. 21, 2005 to Olson,
20040249324, issued Dec. 9, 2004 to Louis,
20040230147, issued Nov. 18, 2004 to Fretterd.

There are also systems involving transmission of energy and allowing a change of massage applicators. Examples of such products are shown in the following U.S. patents:
U.S. Pat. No. 7,354,408, issued Apr. 8, 2008 to Muchisky,
U.S. Pat. No. 7,229,424, issued Jun. 12, 2007 to Jones et al.,
U.S. Pat. No. 7,175,592, issued Feb. 13, 2007 to Lee,
U.S. Pat. No. 6,988,997, issued Jan. 24, 2006 to Stultz,
U.S. Pat. No. 6,758,826, issued Jul. 6, 2004 to Luettgen et al.
U.S. Pat. No. 6,735,808, issued May 18, 2004 to Chen,
U.S. Pat. No. 6,585,668, issued Jul. 1, 2003 to Nissim,
U.S. Pat. No. 6,332,873, issued Dec. 25, 2001 to Naruse et al.,
U.S. Pat. No. 6,267,737, issued Jul. 31, 2001 to Meilus,
U.S. Pat. No. 6,241,693, issued Jun. 5, 2001 to Lambden,
U.S. Pat. No. 5,187,827, issued Feb. 23, 1993 to Wei,
U.S. Pat. No. 4,919,117, issued Apr. 24, 1990 to Muchisky et al.,
U.S. Pat. No. 4,102,334, issued Jul. 25, 1978 to Muchisky,
U.S. Pat. No. 4,098,266, issued Jul. 4, 1978 to Muchisky et al.

There are also devices/systems using gravity as energy for massaging. Examples of such products are shown in the following U.S. patents:
U.S. Pat. No. 7,320,668, issued Jan. 22, 2008 to Warder,
U.S. Pat. No. 6,217,121, issued Apr. 17, 2001 to Mollet,
U.S. Pat. No. 5,913,839, issued Jun. 22, 1999 to Wincek,
U.S. Pat. No. 1,265,083, issued May 7, 1918 to Hoard.

While each of the above-noted developments is useful, there is still a need to reduce physical stress on massage therapists' hands, wrists, arms and shoulders.

### OBJECTS OF THE INVENTION

Accordingly, it is an object of the invention to provide an ergonomic massage tool the use of which is relatively easy on therapist's hands, wrists, arms and shoulders by reducing the stress on and in them while providing an effective massage.

Further objects and advantages of the invention will become apparent from a consideration of the ensuing description and drawings.

### SUMMARY OF THE INVENTION

In accordance with one aspect of the invention, there is provided a massage tool which comprises a shank coupled with a user's body support and with a massage applicator spaced from the body support by a predetermined distance.

In an embodiment of the invention, the massage tool comprises a shank having a predetermined length, the shank having a first end adapted for engaging a user's (massage therapist's) shoulder, upper arm or vicinity, and a distal second end coupled with a massage applicator.

In one embodiment, the first end is coupled with a body engagement structure and the distal end is coupled with a massage applicator (a body contact head).

In one embodiment, the shank comprises means for adjusting its length according to the massage therapist's size, personal preference and ergonomic considerations. The shank may be extendible and/or retractable. In one embodiment, the shank is foldable. In one embodiment, it is made of separable components for easy storage and/or transportation.

In an embodiment of the invention, the shank comprises at least one hand grip portion. The portion may be of a size and shape to facilitate manipulation of the shank during a massage.

In one embodiment, the hand grip portion is disposed at an angle relative to the longitudinal axis of the shank, the angle selected according to ergonomic considerations.

Preferably, the shank is rigid enough to transfer a force from the massage therapist's body to the recipient's body, the force sufficient for massage purposes. The shank may be made of a material exhibiting a degree of flexibility and/or may comprise a compressible mechanism, e.g. a shock absorber, so as to counteract a possibility of the user's excessive force, if any, applied through the user's body during the therapy.

The contact heads may be removable from said distal end of the shank and interchangeable.

According to another aspect of the invention, there is provided a method for performing a massage using a tool comprising a shank having a first end coupled with a user's body support and with a massage applicator spaced from the body support by a predetermined distance, the method comprising the steps of
a) contacting the applicator with the massage recipient's body,
b) applying a pressure with the body through the body support and the shank onto the massage applicator with a force selected to perform a massage therapy, and
c) moving the shank with the applicator on a massage recipient's body to perform the massage.

For the purpose of the present specification, the term “shank” denotes any structure, elongated or not, suitable to be coupled with a body support and a massage applicator.

According to another aspect of the invention, there is provided a method for performing a massage using a tool comprising a shank having a first end coupled with a user's body support and with a massage applicator spaced from the body support by a predetermined distance, the method comprising the steps of
d) contacting the applicator with the massage recipient's body,
e) applying a pressure with the body through the body support and the shank onto the massage applicator with a force selected to perform a massage therapy, and
f) moving the shank with the applicator on a massage recipient's body to perform the massage.

For the purpose of the present specification, the term “shank” denotes any structure, elongated or not, suitable to be coupled with a body support and a massage applicator.

### BRIEF DESCRIPTION OF THE DRAWING FIGURES

Preferred embodiments of the invention will now be described with reference to the accompanying drawings, in which:

FIG. 1.a is a front view of an embodiment of the massage tool;

FIG. 1.b is a front view of another embodiment of the massage tool;

FIG. 1.c is a front view of yet another embodiment of the massage tool;

FIG. 1.d is a front view of yet another embodiment of the massage tool;

FIG. 2a is an illustration of the use of the massage tool of the invention;

FIG. 2b is another illustration of the use of the massage tool;

FIG. 3 is a front view of the body support;

FIG. 4 is a cross-section along the lines A-A of the body support of FIG. 3;

FIG. 5 shows a side view of another embodiment of the tool without a massage applicator;

FIG. 6 shows a sectional front view of an applicator holder;

FIG. 7a is a front view of an exemplary massage applicator;

FIG. 7b is a side view of the applicator of FIG. 7a;

FIG. 8a is a perspective view of another massage applicator;

FIG. 8b shows a front view of the applicator of FIG. 8a;

FIG. 9 is a perspective view of yet another massage applicator;

FIG. 10a is a perspective view of still another massage applicator;

FIG. 10b illustrates a front view of the applicator of FIG. 10a; and

FIG. 11 illustrates still another embodiment of a massage applicator.

### DETAILED DESCRIPTION

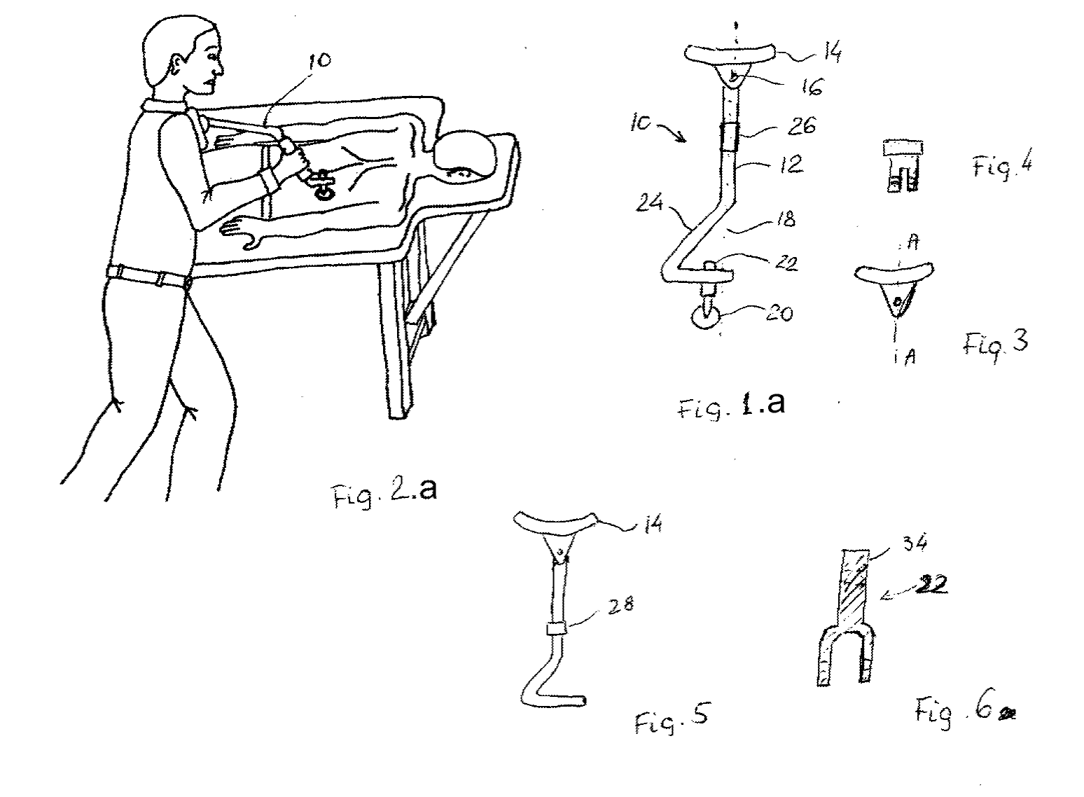

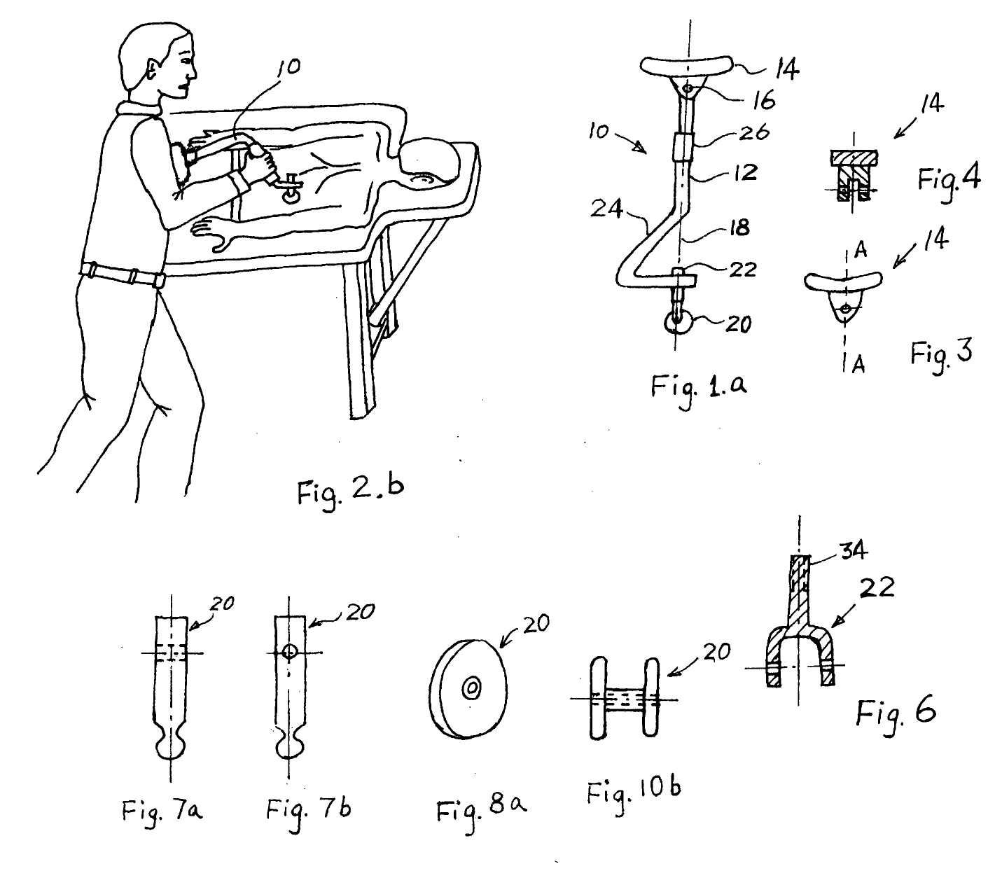

FIG. 1.a illustrates one embodiment of the massage tool of the invention. The tool, generally designated as 10, has a shank 12 which is connected with a body support 14 through a pivotable connection 16 enabling the body support, in operation of the tool, to adapt to the curvature of the user's body. The shank has a longitudinal axis 18. At the end of the shank 12 opposite to the body support, a massage applicator 20 is coupled to the shank 12 using a known mechanical connection. In FIG. 1.a, the applicator is a wheel mounted to a holder 22 which is in turn threadedly connected to the shank 12.

The shank has a grip portion 24 which is bent at an angle to the axis 18 to facilitate hand grip for ergonomic reasons. Alternatively, the shank may be shaped to form two hand grip portions (not illustrated).

The shank should be lightweight to reduce the user's fatigue in operation. It may be made of metal or another material, e.g. a polymer. It may have a degree of flexibility to reduce the possibility of discomfort or injury of the massage recipient in case of application of excessive force by the user of the tool during massage.

The length of the shank 12 can be adjusted to suit the size and preference of the user (distance from the user's shoulder to the massage recipient's body may vary) and/or therapy requirements. The length adjustment means are schematically represented at 26. They may be exemplified by a separable segment of the shank or an extendible portion thereof.

The shank 12 may comprise at least one compressible mechanism, e.g. a well-known spring-loaded shock absorber (not illustrated) so as to counteract a possibility of the user's excessive force, if any, applied through the tool during the therapy.

FIG. 1.b illustrates another embodiment of the massage tool of the invention. The grip portion is placed in between two elongated elements of a crutch-like shank 12 for improved grip or for providing alternative grip options for various users.

FIG. 1.c illustrates another embodiment of the massage tool of the invention. The grip portion is placed at one side of the shank 12 for improved grip or for providing alternative grip options for various users.

FIG. 1.d illustrates another embodiment of the massage tool of the invention. The grip portion is placed at both sides of the shank 12 for improved grip or for providing alternative grip options for various users.

FIG. 2a is an illustration of the use of the massage tool of the invention. A massage therapist (also synonymously referred to as a user in the specification) holds the tool 10 so that one end of the tool (the applicator end) contacts the body of a prone massage recipient and the other end, with a body support, rests against the therapist's shoulder or a shoulder area. The body support may be placed in the armpit, against the shoulder joint, against the upper arm or anywhere in the vicinity, the purpose being for the user to apply a force with the user's upper body onto the applicator end and thereby reduce the energy use by hand and arm muscles. In the FIG. 2a, the body support is shown for simplicity as resting against the shoulder joint of the user. In FIG. 2b, the body support rests against the user's upper arm.

For clarification, the term “body support” denotes herein an element of the tool of the invention that can be shaped and sized to be used as either the top of a crutch (when placed in the armpit of the user) or as a rifle butt (when placed against the shoulder or in the vicinity thereof).

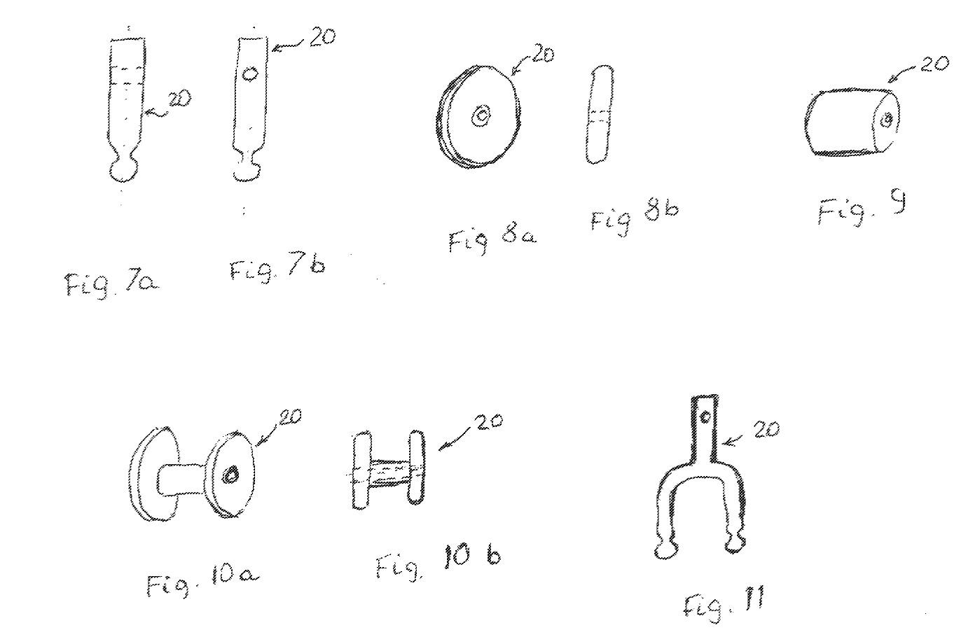

FIGS. 3 and 4 show the body support in more detail.

As shown in FIG. 5, the shank can be assembled from two parts for easy storage and transportation, element 28 denoting a temporary connection e.g. a bayonet connection. Alternatively, the shank may be foldable, wherein the element 28 would denote a suitable joint, known in the mechanical art.

The applicator 20 is mounted on the shank using an applicator holder 22 shown in detail in FIG. 6. The upper part 34 of the holder 30 is threaded into the lower part of the shank 12 (FIG. 1) or connected thereto using alternative suitable means.

FIGS. 7a, 7b, 8a, 8b, 9, 10a, 10b and 11 illustrate various applicators which can be used in connection with the tool of the invention. The applicators may be ball tips, wheels, rollers, contact heads with two contact points (FIG. 11) for areas like the soft tissues along the two sides of the spine, or other massage applicators known in the prior art.

The element in FIG. 9 may be incapable of rolling but has a soft surface to allow user to place their hand, finger or fingers underneath on patient's body to, at one hand, maintain palpation, i.e. feeling the patient's muscles, and on the other hand, applying force from the user's shoulder, armpit, trunk, other upper arm or the palm of the other hand as opposed to from the user's fingers or edges of the hand, hence reduce the stress on the user's fingers and hand. The diameter should be large enough to minimize the pressure on user's hand, finger or fingers, but small enough to be easily manipulate-able. An good compromised diameter would be about 1 inch. The length of the element should be long enough to be able to cover 1 to 4 fingers, and should short enough to be easily manipulate-able. A reasonable compromised length would be 2-3 inches. The soft surface can be made of, for example but not limited to, soft rubber layer, soft polyurethane layer, sturdy foam layer, or any such material know in the art, to protect the user's hand, finger or fingers and to allow greater degree of comfort possible. In such situation, the user will first place one hand, finger or fingers at desired spot on patient's body, then place the soft-surfaced element according to FIG. 9 on the hand, finger or fingers, then apply strength from their upper body such as shoulder, while sensing patient's muscle or muscles with the hand, finger, or fingers on patient body and using the other hand stabilize the massage tool 10 and guide the application of the strength from the user's upper body.

In operation, as illustrated in FIGS. 2a and 2b, the therapist places the massage applicator on desired location of patient's body and applies a suitable amount of pressure onto patient's body by pressing on the body support 14 of the massage tool 10. The therapist guides the shank of the tool with hand and shoulder, while controlling the pressure applied on the body support.

Although the description above contains many specifics, these should not be construed as limiting the scope of the invention but as merely providing illustrations of some of the presently preferred embodiments of this invention. For example, the body support may have other shapes and structure or it may be connected with the shank in a manner allowing the pivotal motion in many planes; the extended rigid structure may have other shapes than illustrated; the applicator connection to the tool may be effected in many ways; the applicator may be integral with the tool; the applicators may have various shapes, dimensions, textures, hardness to suit various therapeutic needs; the shank or other components of the tool may be equipped with oscillatory motor/mechanism to allow massage applicator to vibrate to provide further therapeutic benefits.

Thus the scope of the invention should be determined by the appended claims and their legal equivalents, rather than by the examples given.

1. A massage tool which comprises a shank coupled with a user's body support and with a massage applicator spaced from the body support by a predetermined distance.

2. The massage tool according to claim 1 wherein the shank has a first end coupled with the user's body support, and a distal second end coupled with the massage applicator.

3. The tool according to claim 1 wherein the shank comprises length adjusting means for adjusting the length of the shank according to the massage therapist's size, personal preference or ergonomic considerations.

4. The tool according to claim 1 wherein the shank is foldable.

5. The tool according to claim 1 wherein the shank is made of separable components.

6. The tool according to claim 1 wherein the shank comprises at least one hand grip portion which is sized and shaped to facilitate manipulation of the shank during a massage.

7. The tool according to claim 6 wherein the shank defines a longitudinal axis and the hand grip portion is disposed at an angle relative to the longitudinal axis, the angle selected according to ergonomic considerations.

8. The tool according to claim 1 wherein the shank is made of a material exhibiting a degree of flexibility selected to alleviate excessive force, if any, exerted by the tool user during a massage.

9. The tool according to claim 1 wherein the shank comprises a compressible mechanism exhibiting a degree of flexibility selected to alleviate excessive force, if any, exerted by the tool user during a massage.

10. The tool according to claim 1 wherein the shank comprises an oscillatory mechanism to provide vibratory action of the massage applicator.

11. The tool according to claim 1 wherein the massage applicator has a soft surface suitable for allowing user to place their hand, finger or fingers immediately underneath to feel patient's body directly on the spot of compression while applying compression force from user's trunk, the other arm or the palm of the other hand.

12. A method for performing massage using a tool comprising a shank coupled with a user's body support and with a massage applicator spaced from the body support by a predetermined distance, the method comprising the steps of
a) contacting the massage applicator with the massage recipient's body,
b) applying a pressure with the shoulder through the body support and the shank onto the massage applicator with a force selected to perform a massage therapy, and
c) moving the shank with the applicator on a massage recipient's body to perform the massage.

13. The method according to claim 12 wherein the tool comprises a hand grip portion and the step c) comprises holding the tool at the grip portion.
